(19) United States
(12) Patent Application Publication
(10) Pub. No.: US 2015/0230839 A1
(43) Pub. Date: Aug. 20, 2015

(54) Shark Staple

(71) Applicant: Nicholas Riccione, Slatington, PA (US)

(72) Inventor: Nicholas Riccione, Slatington, PA (US)

(21) Appl. No.: 14/184,861

(22) Filed: Feb. 20, 2014

(51) Int. Cl. A61B 17/80

(52) U.S. Cl. CPC A61B 17/8014

(57) ABSTRACT

An internal fixation comprising an integrated plate with a plurality of pins having double reverse barbs for use in orthopedic surgery to maintain reduction of osteotomy, fracture or arthrodesis site and to provide compression and stability across surfaces of bone for successful ossification and healing is disclosed.

### FIELD OF INVENTION

The present invention relates to an internal fixation widely used in orthopedic surgery to maintain reduction of osteotomy, fracture or arthrodesis site and provide compression and stability across surfaces of bone for successful ossification and healing.

### BACKGROUND OF THE INVENTION

Typically multiple screws and potentially a plate is used in conjunction with screws for orthopedic surgery to maintain reduction of osteotomy, fracture or arthrodesis site and provide compression and stability, especially for procedures on the lower extremities. However, the technique of application for these components are complex, with a steep learning curve and are not without inherent limitations and complications. Practitioners have noticed that in case of lower extremity surgery, when multiple screws with or without a plate, there is rarely a consensus on optimal placement of plate and screws to obtain desired stability and compression.

Also, when using screws without a plate, the screws are never working in tandem but individually. Thus, there exists a problem of non-union, delayed union as well as hardware becoming excessive resulting in stiffness of the foot. In the times of placing bone grafts, screws need to cross the bone graft versus host interface, resulting in significantly diminished surface area contact and therefore delayed healing. Also when placing a screw across any osteotomy or fracture, endosteal blood flow is interrupted as well resulting in delayed healing.

Also, in many different hindfoot and midfoot procedures the actual placement of the screws is difficult because of operating table and patient positioning issues.

Thus, there is a need for a medical device for a lower extremity bone fixation device which provides extra focal compression (much like an external fixator). Further, there is a need for a lower extremity bone fixation device which preserves maximal surface to surface contact of tissue therefore, maximizing the potential for healing of the arthrodesis, osteotomy or fracture.

### DESCRIPTION OF THE PRIOR ART

One example of a prior art approach is shown in the Wright CLAW II polyaxial compression plating system. This system teaches the use of multiple locking screws using a variety of different plate configurations. Such a system does not teach the use of a single, integrated system, nor a standardize plate configuration, and does not address several of the problems which are addressed by the present invention.

Another discussion of a prior art approach may be found in the Integra UNI-CP Plate compression plate system. This system teaches the use of various 2 or 4 screw plate designs, with insertable screws and Surfix® Locking Technology. As with the Wright system, this reference system does not teach the use of a single, integrated system, nor a standardize plate configuration, and does not address several of the problems which are addressed by the present invention.

### DEFINITION OF TERMS

The following terms are used in the claims of the patent as filed and are intended to have their broadest plain and ordinary meaning consistent with the requirements of the law.

A plate refers to a metallic plate used in orthopedics for internal fixation of bone, typically after fractures, the plate being designed to exert pressure between the bone fragments to be transfixed.

As used herein, the term pins refers to metal pins anchored in bone.

Where alternative meanings are possible, the broadest meaning is intended. All words used in the claims set forth below are intended to be used in the normal, customary usage of grammar and the English language.

### OBJECTS AND SUMMARY OF THE INVENTION

The apparatus and method of the present invention generally includes an improved internal compression plate and method of use thereof with lower extremity bone surgical procedures. Specifically, the present invention includes the use of a single, integrated unit compression plate including a plurality (preferably four) pins. The pins being part of a single unit are then functioning simultaneously and synergistically which is different, fundamentally from all other internal fixation options. The pins, in addition to being fixed to the plate, preferably include double reverse barbs also allow for maximal “bite” into bone further increasing the staple's stability and preventing any micromotion subsequent to bone resorption through the course of healing. One template allows to safely and accurately pre drill guide holes for the pins on the plate, coupled with the use of a reverse plier system by the surgeon to facilitate controlled and measured compression according the requirement of the procedure. This preferred feature provides dynamic compression with selective compression of the staple bridges under direct visualization at time of surgery, as opposed to current internal fixation which is limited to “all or none” type of compression. This will reduce the potential for inaccurate placement of the fixation, as well as facilitating appropriate compression/stabilization, ease of application and maximizing bone to bone contact without sacrifice of the interface surface area.

The immediate application of the present invention will be seen in providing an improved internal compression plate that provides extra focal compression (much like an external fixator).

A further object of the present invention is to provide improved internal compression plate that preserves maximal surface to surface contact of tissue therefore, maximizing the potential for healing of the arthrodesis, osteotomy of fracture.

Still another object of the present invention is to provide an improved internal compression plate that offers ease of application by eliminating the need for measuring other than direct visualization of the bone to bone placement

Yet another object of the present invention is to provide an improved internal compression plate that enables improved placement of the fixation, as well as facilitating appropriate compression/stabilization, ease of application and maximizing bone to bone contact without sacrifice of the interface surface area.

It should be noted that not every embodiment of the claimed invention will accomplish each of the objects of the invention set forth above. In addition, further objects of the invention will become apparent based the summary of the invention, the detailed description of preferred embodiments, and as illustrated in the accompanying drawings. Such objects, features, and advantages of the present invention will become more apparent in light of the following detailed description of a best mode embodiment thereof, and as illustrated in the accompanying drawings.

### BRIEF DESCRIPTION OF THE DRAWINGS

FIGS. 1a-d show perspective, top, side and pin detail drawings, respectively, of a contoured, long barb embodiment of the present invention.

FIG. 2a-d show perspective, top, side and pin detail drawings, respectively, of a contoured, short barb embodiment of the present invention.

FIG. 3a-d show perspective, top, side and pin detail drawings, respectively, of a square plate, long barb embodiment of the present invention.

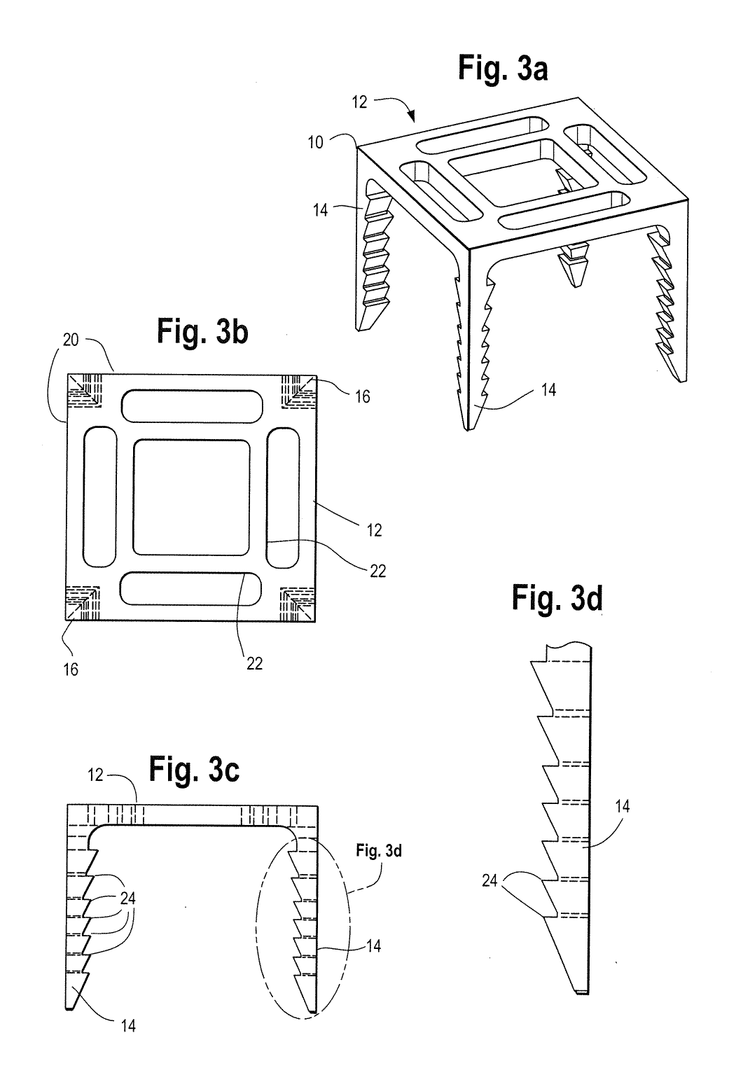

FIG. 4a-d show perspective, top, side and pin detail drawings, respectively, of a square plate, short barb embodiment of the present invention.

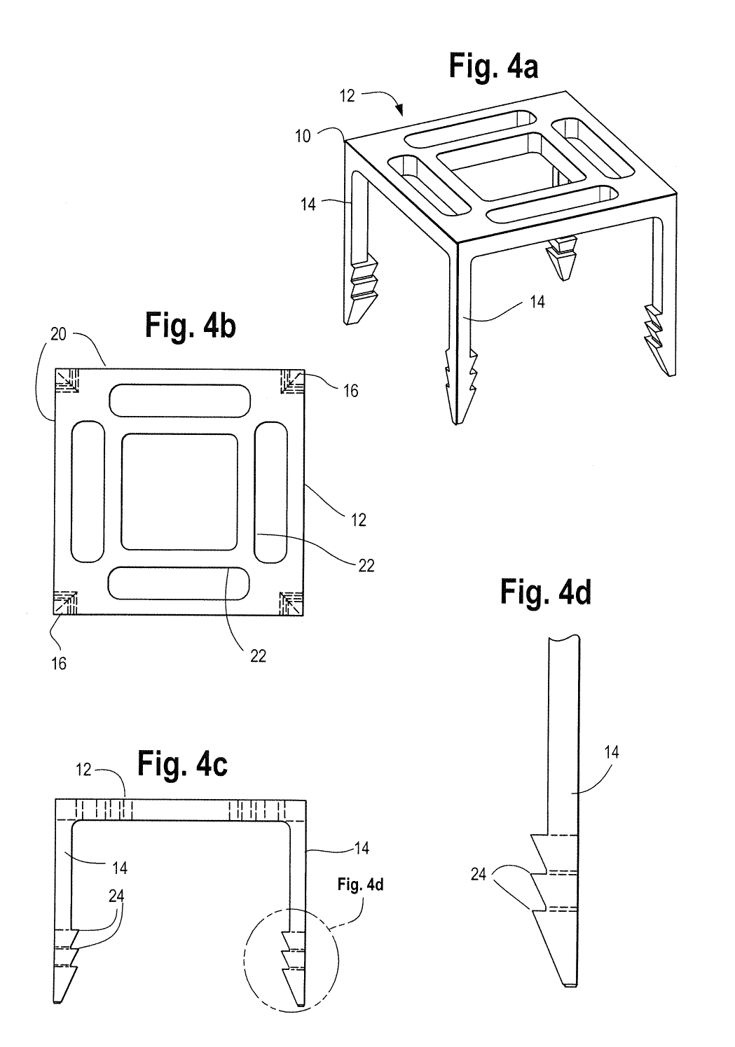

### DETAILED DESCRIPTION OF THE INVENTION

Set forth below is a description of what is currently believed to be the preferred embodiment or best examples of the invention claimed. Future and present alternatives and modifications to this preferred embodiment are contemplated. Any alternatives or modifications which make insubstantial changes in function, in purpose, in structure or in result are intended to be covered by the claims in this patent.

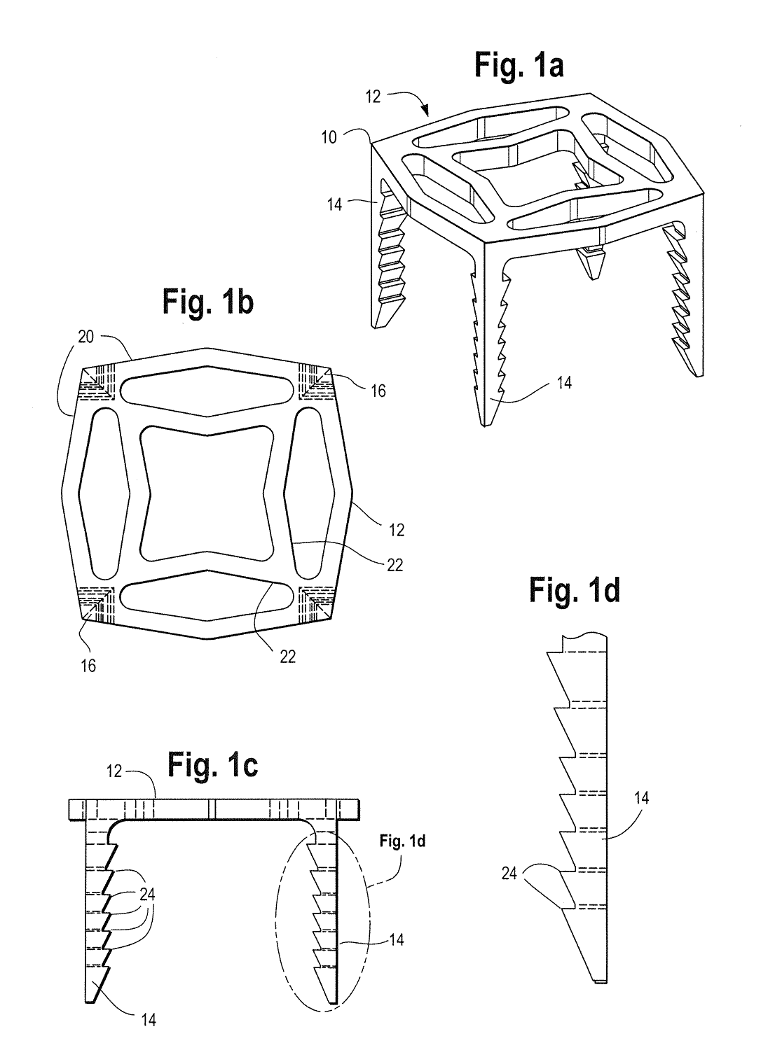

As can be seen in FIGS. 1a-d, a first preferred embodiment of the present invention is a fixture 10, which is comprised of a plate 12 integrally attached to pins 14, each of the pins 14 extending generally orthogonally from the corners 16 of plate 12. The plate further includes external 20 and internal 22 compression actuation surfaces extending between each of the corners. In application, the surgeon can use a reverse plier (not shown) or similar instrument to increase the compression between the various corners 16 so as to increase the compression force exerted by the fixture 10 on the bones. In this first embodiment, the compression force is further may be provided by each of the pins 14, each pin 14 further including a series of barbs 24, specifically double reverse barbs. These barbs 24 can be inserted into holes in the bones that pre-drilled to the specifications of the standardized size of the plate 12, with the barbs also allowing for maximal “bite” into bone further increasing the staple's stability and preventing any micromotion subsequent to bone resorption through the course of healing.

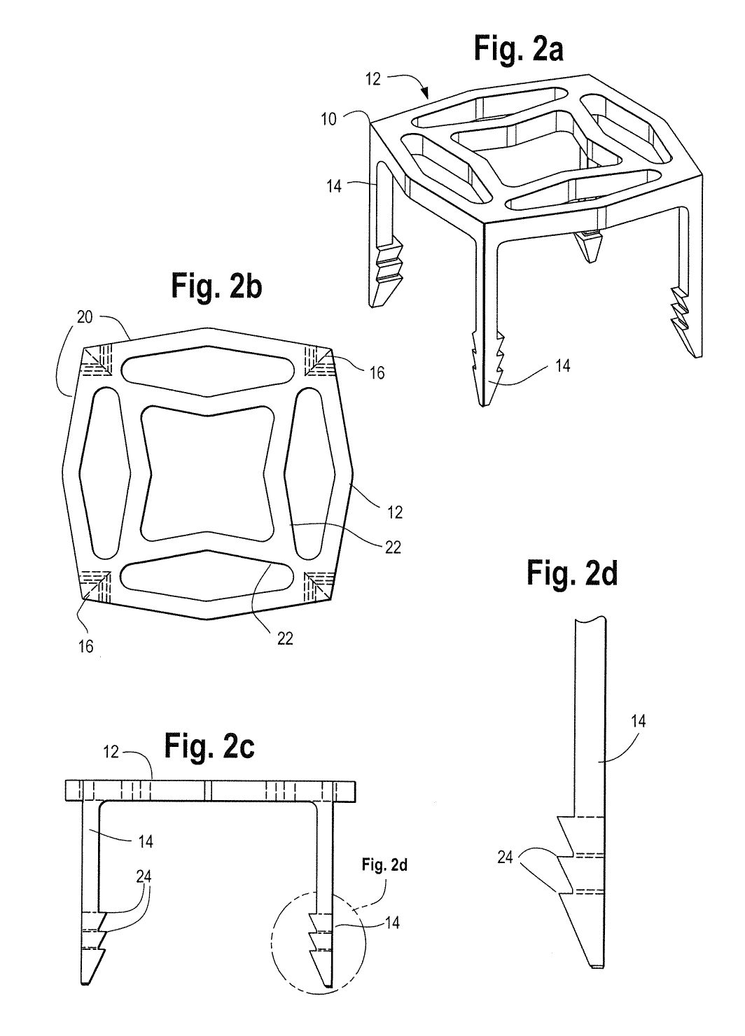

As shown in FIGS. 2a-d, 3a-d and 4a-d, the present invention can employ a variety of length of barbs 24 and further can vary the shape of the external 20 and internal 22 compression surfaces so as to provide a contoured, or a straight, square profile, as needed to account for (among other factors) bone density and the desired compression force. However, in a most preferred embodiment, these plates 12 involve a standardized spacing for the corners 16 such that holes can be predrilled and the fixtures 10 can be “swapped” during the procedure prior to placement without deleterious effect.

The above description is not intended to limit the meaning of the words used in the following claims that define the invention. Rather, it is contemplated that future modifications in structure, function or result will exist that are not substantial changes and that all such insubstantial changes in what is claimed are intended to be covered by the claims. For instance, those of skill will understand that the instant invention can also apply to other designs of plates, as well as various locking mechanisms for the pins integrated with such plates. Likewise, it will be appreciated by those skilled in the art that various changes, additions, omissions, and modifications can be made to the illustrated embodiments without departing from the spirit of the present invention. All such modifications and changes are intended to be covered by the following claims.

I claim:

1. An internal bone fixture assembly comprising:
a) a plate having at least two corners;
b) a plurality of pins integrally formed with the plate, each of the plurality of pins extending from a corner, each of the pins adapted for a gripping engagement with bone upon insertion; and
c) at least one external and at least one internal compression actuation surface extending from each corner, the at least external and at least one internal compression actuation surface positioned to cooperatively and dynamically apply compression force to the bones in which the pins have been inserted.

2. The system of claim 1, wherein each of the pins includes a plurality of barbs to enable the gripping engagement with bone upon insertion.

3. The system of claim 1, wherein the at least external and at least one internal compression actuation surface are straight.

4. The system of claim 1, wherein the at least external and at least one internal compression actuation surface are contoured.

5. An internal bone fixture assembly comprising:
a) a plate having four corners;
b) four pins integrally formed with the plate, each of the four pins extending from a corner, each of the pins adapted for a gripping engagement with bone upon insertion; and
c) four external and four internal compression actuation surfaces connecting each corner, wherein each external compression actuation surface cooperates with an internal compression actuation surface to enable the dynamical application of compression force to the bones in which the pins have been inserted.

6. The system of claim 5, wherein each of the four pins includes a plurality of barbs to enable the gripping engagement with bone upon insertion.

7. The system of claim 1, wherein the four external compression actuation surfaces and four internal compression actuation surfaces are straight.

8. The system of claim 1, wherein the four external compression actuation surfaces and four internal compression actuation surfaces are contoured.

9. An internal bone fixture assembly for insertion into the lower extremity bones, the assembly comprising:
a) a plate having four corners;
b) four pins integrally formed with the plate, each of the four pins extending from a corner, each of the four pins including a plurality of barbs for a gripping engagement with bone upon insertion; and
c) four external and four internal compression actuation surfaces connecting each corner, wherein each external compression actuation surface cooperates with an internal compression actuation surface to enable the dynamical application of compression force to the bones in which the pins have been inserted.
